(19) United States
(12) Patent Application Publication
(10) Pub. No.: US 2015/0119391 A1
(43) Pub. Date: Apr. 30, 2015

(54) ANTI-TUMORAL COMPOSITION COMPRISING THE COMPOUND 1-(6--1,3-BENZOTHIAZOL-2-YL)-3-(2-MORPHOLIN-4-YLETHYL)UREA

(71) Applicant: SANOFI, Paris (FR)

(72) Inventors: Jean-Rene AUTHELIN, Saint Germain Les Arpajon (FR); Tsiala BENARD, Sainte Geneviève des bois (FR); Amandine MATHIEU, Paris (FR); Maria-Teresa PERACCHIA, Paris (FR); Helene GOULAOUIC, Sceaux (FR); Sylvie ASSADOURIAN, Paris (FR)

(21) Appl. No.: 14/593,059

(22) Filed: Jan. 9, 2015

Related U.S. Application Data
(63) Continuation of application No. PCT/EP2013/064741, filed on Jul. 11, 2013.

(30) Foreign Application Priority Data
Jul. 12, 2012 (EP) 12305840.6

(51) Int. Cl. A61K 47/40; A61K 9/08; A61K 31/5377

(52) U.S. Cl. CPC A61K 47/40; A61K 31/5377; A61K 9/08
USPC 514/233.2

(57) ABSTRACT

The present invention concerns an anti-tumoral composition comprising the compound 1-(6-{[6-(4-fluorophenyl)[1,2,4]triazolo[4,3-b]pyridazin-3-yl]sulfanyl}1,3-benzothiazol-2-yl)-3-(2-morpholin-4-ylethyl)urea and its use in the treatment of cancer.

The present invention concerns an anti-tumoral composition comprising the compound 1 -(6-{[6-(4-fluorophenyl)[1 ,2,4]triazolo[4,3-b]pyridazin-3-yl]sulfanyl}-1,3-benzothiazol-2-yl)-3-(2-morpholin-4-ylethyl)urea and its use in the treatment of cancer.

The compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea, called “Compound A” hereafter, is a selective MET receptor tyrosine kinase inhibitor developed for solid tumors for intravenous administration. In vitro it has highly potent anti-proliferative activity (nM) on human Met-driven tumor cell lines exclusively. The cellular anti-tumor activity as a single agent in human Met-amplified models is also highly potent.

This compound and the process for the preparation thereof are described in WO2009/056692.

Compound A is a Biopharmaceutics Classification System (BCS) class IV due to its very poor solubility in water (<0.1 μg/mL) and poor permeability characteristics, thus developed for intravenous administration to overcome the permeability issue.

The development of a stable pharmaceutical composition in the form of an injectable formulation comprising a pharmaceutically acceptable excipient or vehicle and compound A at a dose allowing the clinical use thereof is therefore a challenge.

Moreover, there is still a need to find and optimize new therapeutic options to treat patients with cancer, in particular with advanced solid tumors, such as MET-gene amplified tumors.

The invention meets this need by providing a new pharmaceutical anti-tumoral composition comprising the compound A for which appropriate doses, a suitable administration protocol and/or an acceptable toxicity have been determined. This composition does generally not exacerbate the toxicity of the anti-tumoral agent and allows the treatment of cancer, in particular of advanced solid tumors, such as MET-gene amplified tumors, notably either by stabilizing or by leading to a partial or complete regression of the tumor.

### Concentrated Liquid Aqueous Pharmaceutical Composition

According to a first aspect, the present invention provides a concentrated liquid aqueous pharmaceutical composition (called “concentrated pharmaceutical composition” hereafter) comprising the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea, or a pharmaceutically acceptable salt thereof and a cyclodextrin.

This concentrated liquid aqueous pharmaceutical composition generally is the infusion concentrate.

The present invention is based on the discovery that an aqueous solution of cyclodextrin allows solubilizing the compound A, even at high doses of compound A.

The liquid aqueous composition according to the invention is generally an aqueous solution, i.e. all the components thereof, and notably the compound A, are solubilized in the aqueous solution.

Advantageously, the concentrated pharmaceutical composition is physically and chemically stable and suitable for clinical use, notably in parenteral use, generally after dilution in an appropriate isotonic medium.

In an embodiment, the cyclodextrin of the concentrated pharmaceutical composition is a hydroxypropyl-beta-cyclodextrin (HP-β-CD) or a sulfobutylether-beta-cyclodextrin sodium (SBE-β-CD).

In an embodiment, the cyclodextrin of the concentrated pharmaceutical composition is a sulfobutylether-beta-cyclodextrin sodium (SBE-β-CD), such as Captisol®.

The combined effect of pH and cyclodextrin concentration on compound A solubilization was assessed and the solubility profile of compound A was shown to be strongly dependent of both pH and cyclodextrin concentration.

In an embodiment, in the concentrated pharmaceutical composition, the cyclodextrin concentration is from 10 to 50% w/v, typically around 40% w/v (wherein w/v means weight/volume). These concentrations of cyclodextrin advantageously allow:
a sufficient solubility of compound A at both temperatures 5° C. and 25° C., and
a good processability of the concentrated pharmaceutical composition, in particular an acceptable viscosity for its handling and preparation.

Furthermore, based on compound A concentration in the cyclodextrin solution and the ratio compound A/cyclodextrin, the maximal cyclodextrin dose to be administered is considered as safe and compatible with the compound A dose escalation protocol described hereafter.

As the compound A is a weak base (pKa=6.4), its solubility is pH dependent. For example, its solubility in water is about 90 μg/mL at pH 1, about 20 μg/mL at pH 3 and below 0.1 μg/mL above pH 5.

In an embodiment, the pH of the concentrated pharmaceutical composition is from 2.5 to 4.0. Precipitation of hydrochloride salt can occur by common ion effect at high hydrochloride concentrations below pH 2.0 and the solubility dropped markedly above pH 4.5 due to the intrinsic solubility of compound A.

In an embodiment, the pH of the concentrated pharmaceutical composition is from 2.5 to 3.5, typically around 3.0. Indeed, as described hereafter, the concentrated pharmaceutical composition is a concentrated form which can be diluted, the obtained diluted pharmaceutical composition being the composition which is administered to the patient. One of the encountered challenges of the dilution is to avoid the precipitation of compound A, which could occur due to a lower cyclodextrin concentration and/or an increase of the pH. A pH from 2.5 to 3.5, notably 3.0, advantageously allows dilution of the concentrated pharmaceutical composition without precipitation of compound A in a certain range of concentration of compound A (from around 0.4 to around 5 mg/mL). Indeed, a too low concentration of compound A does generally not allow to obtain a pH compatible with infusion (too acidic pH) and a too high concentration of compound A can lead to the risk of too high hypertonicity of the solution for infusion. The obtained diluted pharmaceutical composition is thus generally physico-chemical stable for at least 24 h at room temperature (around 25° C.).

Accordingly, the concentrated pharmaceutical composition can comprise acids, such as hydrochloric acid, and/or bases, such as sodium hydroxide, in order to obtain the above-described pH.

In an embodiment, in the concentrated pharmaceutical composition, the weight ratio of compound A/cyclodextrin is 1/40.

In an embodiment, in the concentrated pharmaceutical composition, the weight ratio of compound A/cyclodextrin is 1/26.7.

Typically, the concentration of compound A in the concentrated pharmaceutical composition is from 5 to 20 mg/mL, usually from 10 to 15 mg/mL, such as 10 mg/mL or 15 mg/mL. These concentrations are particularly suited to administrate the compound at a dose up to 960 mg/m². In one embodiment, the concentrated pharmaceutical composition is adapted for the administration of the compound A at a dose between 440 and 570 mg/m², advantageously of either 440 or 570 mg/m².

Generally, the water of the concentrated aqueous pharmaceutical composition is water for injection.

In an embodiment, the concentrated liquid aqueous pharmaceutical composition consists in compound A, sulfobutylether β-cyclodextrin sodium, water for injection, hydrochloric acid and sodium hydroxide.

The above-described embodiments can be combined with each other.

In an embodiment, called embodiment 1 hereafter, the concentrated pharmaceutical composition comprises:
10 mg/mL of compound A,
40% w/v of sulfobutylether-beta-cyclodextrin sodium, and its pH is 3.0.

In an embodiment, the unit dose of the concentrated pharmaceutical composition according to embodiment 1 comprises 50 mg of compound A.

In an embodiment, called embodiment 2 hereafter, the concentrated pharmaceutical composition comprises :
15 mg/mL of compound A,
40% w/v of sulfobutylether-beta-cyclodextrin sodium, and its pH is 3.0.

In an embodiment, the unit dose of the concentrated pharmaceutical composition according to embodiment 2 comprises 180 mg of compound A.

The concentrated pharmaceutical composition is generally a sterile, non-pyrogenic, injectable, colorless to pale yellow composition. It can for example be packaged in Type I colorless glass vials fitted with elastomeric closures.

Moreover, the concentrated pharmaceutical composition is generally stable for a minimum of 12 months at 25° C., and for a minimum of 24 months at 5° C.

The concentrated pharmaceutical composition is prepared by usual methods for the preparation of aqueous liquid pharmaceutical composition. Generally, the process comprises a sterilization step, which is typically carried out by sterilizing filtration followed by aseptic filling. Steam sterilization is typically avoided as chemical degradation of compound A and of the cyclodextrin has been observed with this sterilization method.

### Diluted Liquid Aqueous Pharmaceutical Composition

The concentrated pharmaceutical composition described above is a concentrated form, which can be diluted.

Generally, no supersaturation occurs after dilution of the concentrated pharmaceutical composition with the optimized parameters described above (pH, nature and concentration of the cyclodextrin, concentration of compound A, other excipients).

The pharmaceutical composition obtained after dilution (i.e. the diluted liquid aqueous pharmaceutical composition) is the “ready-to-use” composition, i.e. the composition which is administered to the patient. Generally, it is the infusible composition.

The dilution media is generally an infusion media, such as an isotonic infusion media.

Thus, the diluted pharmaceutical composition (called diluted pharmaceutical composition hereafter) comprises the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea, or a pharmaceutically acceptable salt thereof, a cyclodextrin and an infusion media.

The infusion media is typically an aqueous sodium chloride solution or a dextrose solution, such as an aqueous 0.9% sodium chloride solution or a 5% dextrose solution.

Advantageously, the diluted pharmaceutical composition is stable at least 24 hours after the dilution at room temperature.

In an embodiment, the concentration of compound A in the diluted pharmaceutical composition is from 0.35 to 5,5 mg/mL.

In an embodiment, the concentration of compound A in the diluted pharmaceutical composition is from 0.38 mg/mL to 3.33 mg/mL.

In an embodiment, the concentration of compound A in the diluted pharmaceutical composition is from 0.58 mg/mL to 4.77 mg/mL.

In an embodiment, the concentration of compound A in the diluted pharmaceutical composition is from 1.16 mg/mL to 5.09 mg/mL.

In an embodiment, the concentrated pharmaceutical composition comprises 10 mg/mL of compound A and the diluted pharmaceutical composition comprises from 0.38 mg/mL to 3.33 mg/mL of compound A. In an embodiment, the concentrated pharmaceutical composition is the pharmaceutical composition according to embodiment 1 and the diluted pharmaceutical composition comprises from 0.38 mg/mL to 3.33 mg/mL of compound A. Typically, in these embodiments, the infusion media is an aqueous 0.9% sodium chloride solution. For example, the aqueous 0.9% sodium chloride solution (and thus the diluted pharmaceutical composition) is contained in polypropylene or polyolefin/polyamide infusion bags.

In an embodiment, the concentrated pharmaceutical composition comprises 15 mg/mL of compound A and the diluted pharmaceutical composition comprises from 0.58 mg/mL to 4.77 mg/mL of compound A. In an embodiment, the concentrated pharmaceutical composition is the pharmaceutical composition according to embodiment 2 and the diluted pharmaceutical composition comprises from 0.58 mg/mL to 4.77 mg/mL of compound A. Typically, in these embodiments, the infusion media is an aqueous 0.9% sodium chloride solution or a 5% dextrose solution. For example, the aqueous 0.9% sodium chloride solution or the 5% dextrose solution (and thus the diluted pharmaceutical composition) is contained in polypropylene infusion bags.

In an embodiment, the concentrated pharmaceutical composition comprises 15 mg/mL of compound A and the diluted pharmaceutical composition comprises from 1.16 mg/mL to 5.09 mg/mL of compound A. In an embodiment, the concentrated pharmaceutical composition is the pharmaceutical composition according to embodiment 2 and the diluted pharmaceutical composition comprises from 1.16 mg/mL to 5.09 mg/mL of compound A. Typically, in these embodiments, the infusion media is an aqueous 0.9% sodium chloride solution or a 5% dextrose solution. For example, the aqueous 0.9% sodium chloride solution or the 5% dextrose solution (and thus the diluted pharmaceutical composition) is contained in polypropylene or polyolefin/polyamide infusion bags.

These concentrations are particularly suited to administrate the compound at a dose up to 960 mg/m². In one embodiment, the diluted pharmaceutical composition is adapted for the administration of the compound A at a dose of 570 mg/m².

In another embodiment, the diluted pharmaceutical composition is adapted for the administration of the compound A at a dose of 440 mg/m².

The weight of compound A in the unit dose in the concentrated and in the diluted compositions is typically from 20 to 1000 mg, for example 50 mg or 180 mg.

In an embodiment, in the diluted pharmaceutical composition, the weight ratio of compound A/cyclodextrin is 1/40.

In an embodiment, in the diluted pharmaceutical composition, the weight ratio of compound A/cyclodextrin is 1/26.7.

In an embodiment, the pH of the diluted pharmaceutical composition is from 3.5 to 4.0, typically around 4.0.

The above-described embodiments can be combined with each other.

### Concentrated Liquid Aqueous Pharmaceutical Composition for Its Use for the Treatment of Cancer

According to a second aspect, the invention relates to the above described concentrated pharmaceutical composition for its use for the treatment of cancer.

In one embodiment, the above described concentrated pharmaceutical composition for its use for the treatment of cancer is adapted for the administration of the compound A at a dose of 570 mg/m².

In another embodiment, the above described concentrated pharmaceutical composition for its use for the treatment of cancer is adapted for the administration of the compound A at a dose of 440 mg/m².

In an embodiment, the administration of the concentrated pharmaceutical composition is a parenteral administration, such as by perfusion, infusion or injection, typically by intravenous infusion.

### Diluted Liquid Aqueous Pharmaceutical Composition for Its Use for the Treatment of Cancer

According to a third aspect, the invention relates to the above described diluted pharmaceutical composition for its use for the treatment of cancer.

In one embodiment, the above described diluted pharmaceutical composition for its use for the treatment of cancer is adapted for the administration of the compound A at a dose of 570 mg/m².

In another embodiment, the above described diluted pharmaceutical composition for its use for the treatment of cancer is adapted for the administration of the compound A at a dose of 440 mg/m².

In an embodiment, the administration of the diluted pharmaceutical composition is a parenteral administration, such as by perfusion, infusion or injection, typically by intravenous infusion.

Compound A for Its Use for the Treatment of Cancer at a Dose of the Compound Comprised from 25 to 960 mg/m²

According to a third aspect, the invention concerns the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea or a pharmaceutically acceptable salt thereof for its use in the treatment of cancer at a dose of the compound comprised from 25 to 960 mg/m².

In a particular embodiment, the compound A or a pharmaceutically acceptable salt thereof is used in the treatment of cancer at a dose of 570 mg/m².

In another particular embodiment, the compound A or a pharmaceutically acceptable salt thereof is used in the treatment of cancer at a dose of 440 mg/m².

In an embodiment, the cancer is an advanced solid tumor.

In an embodiment, the treated patient has an advanced solid tumor with:
a high total MET protein expression (≧50% of tumor cells with 2+ or 3+ MET protein expression at the membrane level on Immunohistochemistry (IHC) assay) and/or
a MET gene amplification (≧10% of cells with MET fluorescent in situ hybridization (FISH)>4 gene copies and ratio MET/CEP≧2).

In an embodiment, the cancer is a MET-gene amplified tumor.

In an embodiment, the MET-gene amplified tumor (i.e. solid tumor with MET gene amplifications) is an advanced-staged disease chosen from advanced gastroesophageal, advanced non small lung cancer (NSCL), metastatic colorectal, head and neck squamous carcinoma (HNSC), glioblastoma, breast, ovarian and pancreatic cancer. In an embodiment, the MET-gene amplified tumor is an advanced-staged disease such as papillary renal cell carcinoma,

This compound generally allows the treatment of advanced solid tumors either by stabilizing or by inducing a partial or complete regression of the tumor.

An effective quantity of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea or a pharmaceutically acceptable salt thereof is generally administered.

The compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea is generally administered at a dose comprised from 25 to 960 mg/m², typically selected from the following doses: 25, 50, 75, 100, 150, 200, 250, 260, 300, 340, 350, 400, 440, 450, 570, 500, 550, 600, 650, 700, 740, 750, 800, 850, 900, 950 and 960 mg/m², for example selected from the following doses: 260, 340, 440, 570, 740 and 960 mg/m².

In one embodiment, the compound A is administered at a dose of 570 mg/m².

In another embodiment, the compound A is administered at a dose of 440 mg/m².

In an embodiment, the compound is administered parenterally, notably by infusion or injection, typically by intravenous infusion.

The compound is generally formulated as a pharmaceutical composition, which can comprise one or more pharmaceutically acceptable excipient(s). The pharmaceutical composition can be in the form of liquid solutions, suspensions or emulsions.

In an embodiment, the compound is formulated in a liquid composition. Liquid compositions for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers and cyclodextrins may be useful excipients to control the release of the active compounds. Intravenous vehicles can include fluid and nutrient replenishers, electrolyte replenishers, such as those based on

Ringer's dextrose, and the like. Other potentially useful parenteral delivery systems for these active compounds include ethylene-vinyl acetate copolymer particles, osmotic pumps, implantable infusion systems, and liposomes.

In an embodiment, the compound is formulated as the concentrated liquid aqueous pharmaceutical composition described above, in particular the liquid aqueous pharmaceutical composition according to embodiment 2.

In an embodiment, the compound is formulated as the diluted liquid aqueous pharmaceutical composition described above.

Typically, the treatment comprises the steps consisting of:
a) diluting the concentrated liquid aqueous pharmaceutical composition as defined above with an infusion media, such as an aqueous sodium chloride solution or a dextrose solution, in order to obtain the diluted liquid aqueous pharmaceutical composition as defined above,
b) administrating the obtained diluted liquid aqueous pharmaceutical composition as defined above to the patient.

For example, the treatment comprises the steps consisting of:
a) diluting a concentrated liquid aqueous pharmaceutical composition comprising:
   15 mg/mL of compound A,
   40% w/v of sulfobutylether-beta-cyclodextrin sodium, and the pH of which is 3.0 with an infusion media, such as an aqueous sodium chloride solution or a dextrose solution, in order to obtain the diluted liquid aqueous pharmaceutical composition comprising from 0.58 mg/mL to 4.77 mg/mL of compound A and the pH of which is around 4,0,
b) administrating the obtained diluted liquid aqueous pharmaceutical composition as defined above to the patient.

The frequency of administration of the compound is typically once a week.

Typically, the cycle of administration lasts at least 4 weeks, typically 4 weeks. The cycle of administration can be repeated, with or without period of rest (i.e. period without administration of the compound) between two cycles. For example, the compound can be administered weekly during 4 weeks (first cycle), then 7 days of rest, then the compound can be administered weekly during 4 weeks (second cycle). In another example, cycles are repeated without rest. In an embodiment, 2 cycles are repeated without rest. In an embodiment, 4 cycles are repeated without rest.

The above-described embodiments can be combined with each other.

The invention also concerns the use of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea or a pharmaceutically acceptable salt thereof in the manufacture of a medicinal product for the treatment of cancer at a dose of the compound comprised from 25 to 960 mg/m².

In one embodiment, the dose of the compound A is of 570 mg/m².

In another embodiment, the dose of the compound A is of 440 mg/m².

The invention also concerns a method for treating cancer comprising administering to a patient in need thereof the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea or a pharmaceutically acceptable salt thereof at a dose of the compound comprised from 25 to 960 mg/m².

In one embodiment, the dose of the compound A is of 570 mg/m².

In another embodiment, the dose of the compound A is of 440 mg/m².

### Compound A for Its Use for the Treatment of Advanced Solid Tumors

According to a fourth aspect, the invention concerns the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea or a pharmaceutically acceptable salt thereof for its use for the treatment of advanced solid tumors.

In one embodiment, the compound A or a pharmaceutically acceptable salt thereof for its use for the treatment of advanced solid tumors is administered at a dose of 570 mg/m².

In another embodiment, the compound A or a pharmaceutically acceptable salt thereof for its use for the treatment of advanced solid tumors is administered at a dose of 440 mg/m².

In an embodiment, the treated patient has an advanced solid tumor with:
a high total MET protein expression (≧50% of tumor cells with 2+ or 3+ MET protein expression at the membrane level on Immunohistochemistry (IHC)) and/or
a MET gene amplification (≧10% of cells with MET fluorescent in situ hybridization (FISH)>4 gene copies and ratio MET/CEP≧2).

In an embodiment, the cancer is a MET-gene amplified tumor

In an embodiment, the MET-gene amplified tumor (i.e. solid tumor with MET gene amplifications) is an advanced-staged disease chosen from advanced gastroesophageal, advanced non small lung cancer (NSCL), metastatic colorectal, head and neck squamous carcinoma (HNSC), glioblastoma, breast, ovarian and pancreatic cancer. In an embodiment, the MET-gene amplified tumor is an advanced-staged disease such as papillary renal cell carcinoma,

This compound generally allows the treatment of advanced solid tumors either by stabilizing or by inducing a partial or complete regression of the tumor.

The pharmaceutical composition comprising said compound is advantageously well tolerated and does not exacerbate the toxicity of the anti-tumoral agent (i.e. 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea).

Any of the conditions or any of their combinations described above for the treatment of cancer (for example doses of compound, parenteral administration, weekly and/or duration of at least 4 weeks, use of the concentrated or diluted liquid aqueous pharmaceutical compositions described above, excipient(s)) can be applied for the treatment of advanced solid tumors.

The invention also concerns the use of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea or a pharmaceutically acceptable salt thereof in the manufacture of a medicinal product for the treatment of advanced solid tumors.

The invention also concerns a method for treating advanced solid tumors comprising administering to a patient in need thereof compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea or a pharmaceutically acceptable salt thereof.

### Definitions

pharmaceutically acceptable acid: organic or inorganic acid having a low toxicity (see “Pharmaceutical salts” J. Pharm. Sci. 1977, 66, 1-19);
advanced solid tumors: locally advanced or metastatic solid tumors, i.e. tumors which are not operable anymore. Such definition concerns then advanced malignant solid tumors.
“Dose” means the administration dose. The dose is not necessarily the “unit dose”, i.e. a single dose which is capable of being administered to a patient, and which can be readily handled and packaged, remaining as a physically and chemically stable unit dose comprising either compound A itself, or a pharmaceutically acceptable composition comprising compound A and one or more pharmaceutically acceptable excipients, such as the concentrated liquid aqueous pharmaceutical composition described above.
The pharmaceutical compositions of the present invention generally contain a therapeutically effective amount of the active principle. The term “therapeutically effective amount,” as used herein, refers to an amount of the active principle present in the pharmaceutical composition being administered that is sufficient to elicit the desired pharmacological or therapeutic effect(s) and/or to prevent development of or alleviate to some extent one or more of the symptoms of the disease being treated. In determining the effective amount or dose, a number of factors are considered by the attending diagnostician, including, but not limited to: the species of mammal; its size, age, and general health; the specific disease involved; the degree of involvement or the severity of the disease; the response of the individual patient; the mode of administration; the bioavailability characteristics of the preparation administered; the dose regimen selected; the use of concomitant medication; and other relevant circumstances.
All components of the present compositions must be pharmaceutically acceptable. As used herein, a “pharmaceutically acceptable” component is one that is suitable for use with humans and/or other animals without undue adverse side effects (such as toxicity, irritation and allergic response) commensurate with a reasonable benefit/risk ratio.

The conditions of the study described below have to be considered for illustrative purposes only.

The study concerns dose escalation, safety, pharmacokinetic and pharmacodynamic, first in man study, of the compound A single agent administered as slow intravenous infusion in adult patients with advanced malignant solid tumors

In the dose escalation part: among the preferred patients (pts) are pts with MET gene amplification and/or high total MET protein expression, evaluable or measurable solid tumors for which no standard therapy is available.

In the expansion cohort, among the preferred patients (pts) are pts with diagnosed MET-gene amplified and measurable tumors

A purpose of such study is in particular, to evaluate the preliminary anti-tumoral effect of compound A in patients (pts) with MET-gene amplified tumors. Then among the preferred patients are any patients with advanced solid tumor diagnosed with MET gene amplification.

Methods of determining the levels of total Met protein expression are known by the man skilled in the art and available commercially.

CMet gene copies (Met gene amplification) are determined using for example FISH (Fluorescence in situ hybridization) as known by the man skilled in the art using for example the following references:
B J Trask—Genome analysis: A laboratory manual, 1999
C M Price—Blood reviews, 1993—Elsevier
J K Blancato—The Principles of Clinical Cytogenetics, 1999
T Haaf—2000—Wiley Online Library
H Goker, J Shipley—Methods in molecular medicine, 2001
B A Tate . . . —METHODS IN MOLECULAR BIOLOGY-CLIFTON , 2002—Springer
B J Trask—Trends in Genetics, 1991—Elsevier

### Study Objectives

### Primary Objective:

### Dose Escalation

To determine the maximum tolerated dose (MTD) of compound A according to the Dose Limiting Toxicity(ies) (DLTs) observed in Asian patients with advanced solid tumors.

### EXPANSION Cohort

To evaluate the preliminary anti-tumoral effect of compound A in patients with measurable MET-gene amplified tumors treated at the recommended dose (RD) of compound A.

### Secondary Objectives:

To characterize and confirm the global safety profile of compound A including cumulative toxicities.
To assess preliminary antitumor activity of compound A in patients with measurable disease, according to RECIST 1.1 criteria.
To explore the pharmacodynamic effects (PDy) of compound A.
To evaluate the pharmacokinetic profile of compound A.
To explore the relationship of MET gene amplification status with anti-tumor effects.
To evaluate other pharmacodynamic biomarkers and help selection of patients who could benefit from compound A, as an exploratory objective.
To evaluate volumetric tumor response as an exploratory objective at the recommended dose.

### Study Design

This is an open-label dose escalation Phase 1 study of safety, pharmacokinetic (PK) and pharmaco-dynamic (PDy) of compound A administered as a weekly intravenous infusion to Asian adult patients with advanced malignant solid tumors. Four weekly administration of compound A are considered as 1 cycle (1 theoretical cycle includes day (D) 1, D8, D15 and D22 infusions=4 weeks).

### Dose Escalation:

The starting dose is a 260 mg/m².

Dose escalation beyond the starting dose is expected to proceed according to the table below.

Dose levels tables

| Dose level | Dose of Compound A | Increment |
|---|---|---|
| (DL) | (mg/m²) | (%) |
| 1 | 260 | 30 |
| 2 | 340 | 30 |
| 3 | 440 | 29 |
| 4 | 570 | 30 |
| 5 | 740 | 30 |
| 6 | 960 | 30 |

A Study Committee is set up, including at least the Investigators, Sponsor team members and ad hoc experts (biomarkers, PK and statistic representatives) when appropriate.

The study committee decides to escalate (or not) dose level during study committee meetings on the basis of their knowledge on the whole safety profile and on the Bayesian design recommendation described hereafter.

An adaptive Bayesian design with overdose control is used to provide dose recommendations on compound A dose escalation.

This adaptive escalation is based on a statistical (two parameter logistic) model for the probability of DLT in the whole population as a function of dose. The model is used to estimate whether the probability of DLT (also called DLT rate) at each candidate dose level is within a targeted interval of 20% to 35% after each new cohort of DLT evaluable patients.

Dose escalation is indicated by the model if the probability of DLT within the targeted interval at the next level is greater than at the current level. Dose de-escalation is indicated if the probability of DLT within the targeted interval at a lower level is greater than at the current level. Otherwise, subsequent patients are treated at the current dose level.

In addition, escalation only occurs when the overdosing risks in the global population and in each sub populations are controlled, that is to say, the risks of a DLT rate above 35% and above 60% should not exceed pre-specified tolerated risk levels.

Enrolment at the next dose level does not proceed before at least 3 patients treated at the current dose level have been followed for at least 4 weeks.

At least 3 patients are treated and evaluated for the Dose Limiting toxicities at each dose level with at least one patient of each country. As much as possible, the numbers of evaluable patients from each country are the same (a country should not be represented by less than 33% of patients at any dose level). An exploratory analysis is built to assess whether the probability of DLT at the selected dose is equivalent for Japanese and Korean patients.

During the dose escalation step a minimum of 1 week is mandatory between patient #1 and patients #2/#3 treated at the same DL. A minimum of 4 weeks elapses between the last patient who has received D1 of cycle 1 (i.e. DLT observation period) at DL n, and the first patient who receives D1 of cycle 1 at DL n+1.

The MTD is defined as the dose having the highest probability to be in the targeted interval (i.e. to generate between 20 and 35% of DLT in the whole Asian population), and verifying overdosing constraints (in the global population and in each country stratum). Estimate of MTD for each ethnicity (Japanese and Korean) is explored on the basis of gathered data.

Although the dose escalation process is guided by the safety evaluation during Cl of treatment, cumulative toxicities observed after subsequent administrations are also considered for the dose escalation and the dose selection decision (i.e. expansion of a given dose level, intermediate dose levels . . . ), upon recommendation from the Study Committee.

The recommended dose (RD) for the expansion cohort is primarily based on safety data. However, especially in case of MTD cannot be determined in absence of DLT at the maximal administrated dose, PK and PDy results can support the determination of the RD.

### Expansion Cohort:

Approximately 15 patients with MET-gene amplified tumor are treated at the RD of compound A to collect safety, PK, PDy and preliminary efficacy data on both Japanese and Korean populations.

Each country should be equivalently represented (no less than 33% of patients from one country) in the expansion cohort. In this cohort, best effort are done to enrol patients with tumor accessible for biopsies and agree to pre-treatment and ontreatment tumor biopsies. The safety is closely evaluated; especially any cumulative toxicity is detected.

Best efforts are done to collect paired biopsies (fresh tumor samples) in at least 1 patient at each DL from DL 260 mg/m² and at least in 5 patients (33%) in the expansion cohort.

### Study Population

### Main Inclusion Criteria:

I 01. From Dose level 260 mg/m²: Solid tumor patients with measurable or non measurable disease for whom no standard therapy is available and high total MET protein expression (≧50% of 2+ or 3+ positive membrane stain on IHC) and/or MET gene amplification (≧10% of cells with MET FISH>4 gene copies and ratio MET/CEP≧2). Archival tumor tissue (FFPE and/or fresh/frozen tissues) samples should have been collected within 12 months prior to study entry and should be available before study entry for a central review.
I 02. At the recommended dose (expansion cohort): only patients with measurable disease and MET gene amplification. Archival tumor tissue (FFPE and/or fresh/frozen tissues) samples should have been collected within 12 months prior to study entry and should be available before study entry for a central review.
03. Patient understands and has signed the Written Informed Consent form and is willing and able to comply with the requirements of the trial.

### Main Exclusion Criteria:

### a) Methodology Related Such As:

E 01. Patient less than 20 years old
E 02. ECOG performance status >2
E 03. Any serious active disease or co-morbid condition, which, in the opinion of the investigator, may interfere with the safety or the compliance with the study
E 04. Poor bone marrow reserve as defined by absolute neutrophils count <1.5×109/L or platelets <100×109/L
E 05. Poor organ function as defined by one of the following:
   Total bilirubin >1.5×ULN
   AST, ALT, alkaline phosphatase >2.5×ULN or >5×ULN in case of documented liver metastasis
   Serum creatinine >1.5×ULN, or serum creatinine between 1.0 and 1.5×UNL associated with calculated creatinine clearance <60 mL/min
   Proteinuria >500 mg/24 h
E 06. Pregnant or breast-feeding women. Sexually active (males and females) who do not agree to use medically acceptable methods of contraception during the course of the study and for 3 months following discontinuation of study drug. Female patients of childbearing potential must have a negative pregnancy test at screening
E 07. No tumor lesion (either measurable or non measurable) in the Dose Escalation part, and no measurable disease in the expansion cohort
E 08. Known brain metastasis (other than totally resected or previously pre-irradiated and no progressive/relapsing) or lepto-meningeal carcinomatosis
E 09. No resolution of any specific toxicities (excluding alopecia) related to any prior anti-cancer therapy to grade ≦1 according to the NCI CTCAE v.4.03
E 10. Wash out period of less than 3 weeks from previous antitumor therapy or any investigational treatment, (and less than 6 weeks in case of prior nitrozo-urea and or mitomycin C treatment)
E 11. Any surgery with major risk of bleeding or wound healing default risk performed less than 10 days prior to study treatment administration
E 12. Any other severe underlying medical conditions, which could impair the ability to participate in the study or the interpretation of its results
E 13. Patients treated with potent CYP3A inhibitor (Cyclosporine, chloramphenicol, troleandomycin, telithromycin, clarithromycin, grapefruit juice, ketoconazole, nefazodone, itraconazole, ritonavir.)
E 14. Patients treated with CYP3A inducers (Amobarbital; carbamazepine; dexamethasone; efavirenz; modafinil; nevirapine; norethindrone; oxcarbazepine; phenobarbital; prednisolone; phenytoin; primidone; rifabutin; rifampin; rifampicin; rifapentin; ritonavir; secobarbital; St John's wort; Troglitazone.)

### b) Related to the Compound A:

E 15. Known hypersensitivity or any adverse event related to the study drug excipient (Captisol®)
E 16. Prior treatment with any MET inhibitor compound (selective or not)

### Total Expected Number of Patients:

It is anticipated that approximately 60 safety-evaluable patients are entered into the study.

Most likely around 25 to 45 safety evaluable patients enrolled during the escalation part, followed by 15 additional evaluable patients enrolled at the recommended dose.

The actual sample size varies depending on DLTs observed and number of dose levels actually explored.

### Expected Number of Sites:

2 sites in dose escalation part, approximately 4 sites in the expansion cohort (patients with MET-gene amplified tumors)

### Investigational Product(S)

### Formulation(s):

### Formulation(s):

Compound A is supplied as a sterile, colorless to pale yellow colored, solution concentration of 15 mg/mL with 40% w/v of sulfobutylether-beta-cyclodextrin sodium. The compound A/SBEβCD w/w ratio is 1/26.7 (wherein w/w means weight/weight). The pH of the solution is 3.0.

This is concentrated solution which is a sterile, colorless to pale yellow solution in 15 mL glass vials fitted with rubber closures and crimped with an aluminum cap and a green plastic lid. Each vial contains 180 mg of compound A and 4.8 g of sulfobutylether-β-cyclodextrin (SBE-β-CD).

The highest potential dose of 960 mg/m² corresponds to a quantity of 25.6 g/m² of SBEβCD (51.2 g Captisol® for a 2 m² BSA or 56.32 g for 2.20 m² BSA) since the compound A/SBEβCD ratio w/w is 1/26.7.

### Storage and Preparation:

Clinical supplies as packaged are stored between 2-8° C.

The content of the vial are further diluted, prior to infusion, with sodium chloride 0.9% or dextrose 5% for injection. A diluted solution comprising from 0.58 to 4.77 mg/mL of compound A is thus obtained.

### Route(s) of Administration:

Slow Intravenous infusion. Duration of infusion ranges from 1.5 hour to 4 hours for the highest dose levels.

### Dose Regimen/Duration:

Compound A is administered every week without rest (D1, D8, D15, D22; D1 of subsequent cycle =D29 of previous cycle). One cycle corresponds to 4 weekly administrations.

Intra-patient dose escalation is not permitted.

Dose reduction, omission and/or treatment delay and/or treatment discontinuation are planned in case of severe toxicity.

Study treatment may continue until disease progression, unacceptable toxicity or patient willingness to discontinue or for a maximum of 1 year after the second cut-off date, if the patient benefits from the study treatment.

### Primary Endpoint(S) and Main Secondary Endpoint(S)

### Primary Endpoint:

Define IMP DLTs observed at first cycle.

Maximal Tolerated Dose (MTD) based on the assessment of DLTs.

Safety is assessed based on physical examination (preferably by the same physician in each specific center), laboratory tests, and reports of adverse events.

DLTs are defined as any of the following AEs during the first cycle of study treatment:
1. Grade 4 neutropenia for 7 or more consecutive days
2. Febrile neutropenia (Grade 3 or 4 neutropenia complicated by fever >38.3° C. or a sustained temperature of >38 degrees C. [100.4 degrees F.] for more than one hour) or neutropenic infection (Grade 3 or 4 neutropenia complicated by infection clinically or microbiologically documented)
3. Grade 4 thrombocytopenia
4. Grade 3 thrombocytopenia with bleeding requiring transfusion
5. Any Grade 3 or higher clinical adverse event
6. Any Grade 3 or 4 non-hematological laboratory abnormalities that are not easily managed or corrected by medical intervention (e.g., administration of concomitant medications, correction of electrolyte abnormalities)
7. Toxicity related to compound A leading to omission of 2 compound A doses (=a delay of more than 2 weeks between two compound A administrations or a period of 3 weeks or more between 2 compound A administrations instead of 1 week) , due to absence of recovery to baseline or <=grade 1 (except for alopecia).

These AEs are considered as IMP related in absence of clear evidence to the contrary and if not related to disease progression, grading using NCI-CTC AE scale (version 4.03). In case of existing usable prophylactic and/or curative treatment for a dose limiting non hematologic adverse event (eg: diarrhea, hypersensitivity, nausea-vomiting, hyperglycemia), this treatment may be evaluated in an additional cohort of up to 6 patients treated at the same dose level. Further dose escalation could be allowed and is performed as explained above (in study design section), on the basis of the DLT observed on this additional cohort receiving the prophylactic/corrective therapy. These prophylactic/corrective therapies are systematically implemented in further dose levels.

In addition, in the expansion cohort, cumulative toxicities are specifically detected, in order to confirm the feasibility of the dose selected in the dose escalation part.

### Secondary Endpoints:

PK parameters of compound A in blood (DBS), i.e. Cmax, AUCs, t1/2z, CL, Vss after single and repeated administration. A blood sample is collected at baseline for Genotyping on CYPs.
Biological Evaluation:
1) biomarkers for patient selection: Total MET expression when DLs≧260 mg/m2 (in dose escalation) and MET gene amplification (in expansion cohort) on tumor tissue
2) biomarkers for PDy effect:
   circulating Shed MET and HGF in plasma,
   Total MET/Phospho-MET expression on tumor tissue,
   MET inhibition related-RNA signature in hair follicles (in expansion cohort only). 3) exploratory tests:
   more exploratory analysis may be performed, in order to correlate the clinical outcome of patients with other biomarkers, such as plasma cytokine biomarkers to determine MET inhibition signature, mutational and/or activation status of various pathways (ex. RAS/MAPK and PI3K/AKT) or various receptor tyrosine kinases (ex. EGFR, HER3, HER2)

When possible, pharmacodynamic sample collection coincides with scheduled PK time points.
Efficacy parameters:

Tumor measurements (CT scans or MRI) are done at baseline, end of cycle 1 in order to detect early response and to evaluate any correlation between imaging and PDy results, and then every 2 cycles. Imaging should be available for a central review upon Sponsor request. A central review of volumetric response is performed for exploratory purpose.

### Assessment Schedule

### SAFETY Evaluation

Vital signs, physical examinations, ECOG PS, chest X-ray, 12-lead ECG, laboratory safety tests (including complete blood counts, serum biochemistry and urinalysis) and ophthalmology tests are obtained prior to study drug administration and at designated intervals throughout the study. Adverse events (AEs) are collected from the signing of the study main informed consent up to 30 days after the last IMP administration. AEs are graded according to the National Cancer Institute Common Terminology Criteria for Adverse Events, Version 4.03 (NCI CTCAE v.4.03) and coded according to medDRA. During the follow-up period, ongoing SAEs regardless of relationship to IMP and ongoing or new study treatment related AEs are followed until resolution or stabilization.

### Pharmacokinetics Assessment Schedule

### Compound A

### Cycle 1:

PK evaluation is performed in all patients for compound A at Day 1 (1st administration) and Day 22 (4^{th} administration). Blood samples (1.2 mL each) for PK analysis are collected at mid-infusion (time depends on the length of infusion), 5 min before the end of infusion (EOI) and then at regular time points at D1, D2, D3, D4, D6 and D8. Additional samples are drawn before start of infusion (predose) on D15 (3^{rd} administration), on D22 (4th administration) and D29 (=D1 C2).

From cycle 2 to cycle 4:

blood samples (1.2 mL each) are collected immediately before the start of infusion (pre-dose) on D1, D8, D15 and D22.

Exploratory assessment of compound A unchanged compound in urine is performed, in the expansion cohort, by an exploratory assay method. Urine is collected at selected interval pre-dose and on Day 1 of Cycle 1.

Blood samples (3mL each) for Captisol® pharmacokinetic analysis are collected in all patients at Cycle 1 at designated time points.

### Genotyping

A blood sample (6 mL) per patient is collected on D1 (predosing) to enable investigation of allelic variants of drug metabolism enzymes (including CYP2D6).

A blood sample (10 mL) is collected on D1 (pre-dosing) as a source of normal DNA for the analysis of genetic variants identified in tumor tissue by genotyping or sequencing studies in all patients.

### Pharmacodynamics Assessment Schedule

Blood samples for the determination of shed MET and HGF in plasma are collected on Cycle 1 D1 and D22 before the start of infusion (pre-dose), and at designated time points. During the escalation part, total-MET and phospho-MET determination are performed in tumor tissue collected at cycle 1 before compound A first infusion and at 48 hours after the fourth compound A infusion. In the expansion cohort, total-MET and phospho-MET determination are performed in tumor tissue collected at cycle 1 before compound A first infusion and at 96 hours after the fourth compound A infusion. Best effort is done to collect tumor tissue at time of disease progression in case of CR or PR or SD has been lasted for at least 4 months.

In expansion cohort: MET inhibition related-RNA signature in plucked hair follicles is established at cycle 1 before compound A first infusion, and 5 min before EOI, 5 hours and at 96 hours after the fourth compound A infusion.

### Antitumoral Activity Evaluation

Antitumor activity is assessed according to RECIST 1.1 by computerized tomography (CT) or MRI and other exams as clinically indicated to assess target and non-target lesions. Volumetric response evaluation is assessed by CT scan.

These exams are performed at baseline (screening), at end of C1, and then every 8 weeks (2 cycles), and whenever disease progression is suspected, using the same method(s) for each assessment.

### Statistical Considerations

### Determination of the Sample Size

The number of dose levels examined and the emerging compound A related toxicities determine the sample size.
According to simulations of various scenarios, it is anticipated that a mean of 25 to 45 patients is required to establish the recommended dose of compound A.
Up to 15 patients are registered in an expansion cohort of patients treated at the MTD.

### General Statistical Approach

Dose Escalation Part

Safety and pharmacokinetic evaluations are performed on the all treated population, defined as all patients exposed to at least one dose of investigational medicinal product. These data are descriptively summarized for each dose level.

Investigational medicinal product (IMP) related DLTs occurring at cycle 1 and adverse events meeting DLT criteria occurring at any other cycle are assessed and analyzed on all treated patients.

To be evaluable in dose escalation, a patient should have received a first complete cycle (4 infusions), unless he/she discontinued the IMP before cycle 1 completion for a DLT. A patient who discontinues the IMP before the end of cycle 1 for a reason other than DLT is replaced.

Type, frequency, seriousness and relatedness of IMP emergent adverse events (TEAEs) are analyzed. TEAEs are analyzed according to MedDRA (Medical Dictionary for Regulatory Affairs).

Laboratory abnormalities are analyzed according to the NCICTCAE v. 4.03.

Pharmacokinetics parameters are summarized with descriptive statistics (mean, geometric mean, median, standard deviation, standard error of the mean, coefficient of variation, minimum and maximum).

Dose proportionality is assessed using a power model on Cmax, AUCO-168, AUClast and AUC. Dose effect is assessed using a linear fixed effects model on log-transformed t1/2z. Time-to-steadystate is estimated by fitting Ctrough values with a nonlinear mixed effects model. Accumulation ratio (D22/D1) for Cmax and AUCO-168 is estimated with 90% Cl using a linear fixed effects model on log transformed parameters.

Preliminary efficacy is descriptively presented on activity/efficacy population defined as all registered patients who have received at least one cycle of the investigational drug, and provide a baseline and at least one post-baseline assessment for the efficacy variable of interest. Patients with an early progression as per RECIST 1.1 are also included in this set.

### Expansion Cohort Part

Similar analyses as for dose escalation part are performed.

### Final Analysis

At the end of the study, a Bayesian analysis allows the estimation of the probability of DLT at the selected dose in each sub population (Japanese and Korean). By this way, the impact of the ethnic factor on the probability of DLT could be explored at the selected dose level.

### Duration Of Study Period

### Per Patient

The duration of the study for one patient includes a period for inclusion of up to 3 weeks and a 4-week treatment cycle(s). The patients may continue treatment until disease progression, unacceptable toxicity or willingness to stop.

### Per Study

The expected enrolment period is approximately 28 months.

The first trial cut-off date is 8 weeks after the last patient treated in the dose escalation part in order to have at least 2 evaluable cycles for all patients; the second study cut-off date is 5 months (4 evaluable cycles and 30-day follow-up period) after the last patient treated in the expansion cohort to better detect any cumulative toxicities and to assess preliminary antitumor activity.

If a patient treated in dose escalation part or in an expansion cohort continues to benefit from the treatment after the second study cutoff date, the patient can continue study treatment for a maximum of 1 year and continues to undergo all assessments as per the study flowchart. Such patients are followed at least until 30 days after the last IMP administration and the following information are collected: IMP(s) administration, IMP related AEs, any SAEs.

What is claimed is:

1. A concentrated liquid aqueous pharmaceutical composition comprising the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea, or a pharmaceutically acceptable salt thereof, and a cyclodextrin.

2. The concentrated liquid aqueous pharmaceutical composition according to claim 1, wherein the cyclodextrin is a sulfobutylether-beta-cyclodextrin sodium.

3. The concentrated liquid aqueous pharmaceutical composition according to claim 1, wherein the cyclodextrin concentration is from 10 to 50% w/v.

4. The concentrated liquid aqueous pharmaceutical composition according to claim 1, the pH of which is from 2.5 to 4.0.

5. The concentrated liquid aqueous pharmaceutical composition according to claim 1, wherein the concentration of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea, or a pharmaceutically acceptable salt thereof, is from 5 to 20 mg/mL.

6. The concentrated liquid aqueous pharmaceutical composition according to claim 1, comprising:
10 mg/mL of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea, or a pharmaceutically acceptable salt thereof, and
40% w/v of sulfobutylether-beta-cyclodextrin sodium, and having a pH of 3.0.

7. The concentrated liquid aqueous pharmaceutical composition according to claim 1, comprising:
15 mg/mL of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea, or a pharmaceutically acceptable salt thereof, and
40% w/v of sulfobutylether-beta-cyclodextrin sodium, and having a pH of 3.0.

8. A diluted liquid aqueous pharmaceutical composition comprising the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea, or a pharmaceutically acceptable salt thereof, a cyclodextrin and an infusion media.

9. The diluted liquid aqueous pharmaceutical composition according to claim 8, wherein the weight ratio of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea/cyclo-dextrin is 1/40.

10. The diluted liquid aqueous pharmaceutical composition according to claim 8, wherein the weight ratio of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea/cyclo-dextrin is 1/26.7.

11. A method for treating cancer comprising administering to a patient in need thereof the concentrated liquid aqueous pharmaceutical composition according to claim 1.

12. A method for treating cancer comprising administering to a patient in need thereof the diluted liquid aqueous pharmaceutical composition according to claim 8.

13. A method for treating cancer comprising administering to a patient in need thereof the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethy)urea, or a pharmaceutically acceptable salt thereof, at a dose of the compound comprised from 25 to 960 mg/m².

14. The method according to claim 13, wherein the dose of the compound is of 570 mg/m².

15. The method according to claim 13, wherein the dose of the compound is of 440 mg/m².

16. The method according to claim 13, wherein the cancer is an advanced solid tumor.

17. A method for treating advanced solid tumors comprising administering to a patient in need thereof a therapeutically effective amount of the compound 1-(6-{[6-(4-fluorophenyl)[1,2,4]triazolo[4,3-b]pyridazin-3-yl]sulfanyl}-1,3-benzothiazol-2-yl)-3-(2-morpholin-4-ylethyl)urea or a pharmaceutically acceptable salt thereof.

18. The method according to claim 13, wherein the compound is parenterally administered.

19. A method for treating cancer comprising:
a) obtaining a concentrated liquid aqueous pharmaceutical composition as defined in claim 1,
b) diluting the concentrated liquid aqueous pharmaceutical composition with an infusion media, in order to obtain a diluted liquid aqueous pharmaceutical composition, and
c) administering the obtained diluted liquid aqueous pharmaceutical composition to a patient in need thereof.

20. The method according to claim 13, wherein the compound is administered once a week.
